(19) United States
(12) Patent Application Publication
(10) Pub. No.: US 2008/0213810 A1
(43) Pub. Date: Sep. 4, 2008

(54) Screening Method for Hiv Rt Inhibitors

(76) Inventors: Dirk Edward Desire Jochmans, Herent (BE); Piet Tom Bert Paul Wigerinck, Terhagen (BE)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/813,817

(22) PCT Filed: Feb. 6, 2006
(86) PCT No.: PCT/EP2006/050707

§ 371 Date: Jul. 12, 2007

(30) Foreign Application Priority Data
Feb. 4, 2005 (EP) 05100809.2
Jun. 1, 2005 (EP) 05104764.5

(51) Int. Cl. C12Q 1/48

(52) U.S. Cl. 435/15

(57) ABSTRACT

The present invention is directed to methods for identifying a specific class of inhibitors of HIV reverse transcriptase that act differently from known reverse transcriptase inhibitors. In particular, the invention is based on identifying inhibitors which have higher inhibitory activity in presence of a nucleoside triphosphate or pyrophosphate.

### FIELD OF THE INVENTION

The present invention is directed to methods for identifying a specific class of inhibitors of HIV reverse transcriptase that act differently from known reverse transcriptase inhibitors.

### BACKGROUND OF THE INVENTION

Drugs that are currently on the market or under development to combat HIV viral infection belong to classes such as reverse transcriptase inhibitors (RTIs), protease inhibitors (PIs) and the more recent fusion inhibitors. RTIs prevent viral replication by intervening in the reverse transcription mechanism while PIs intervene in the viral assembly. RT inhibitors interact with the RT enzyme in a number of ways to inhibit its functioning so that viral replication becomes blocked. PIs bind to the active site of the viral protease enzyme, thereby inhibiting the cleavage of precursor polyproteins necessary to produce the structural and enzymatic components of infectious virons.

Nucleoside Reverse Transcriptase Inhibitors (NRTIs) are a class of RT inhibitors that are intracellularly converted to nucleoside triphosphates that compete with the natural nucleoside triphosphates for incorporation into elongating viral DNA by reverse transcriptase. Chemical modifications that distinguish these compounds from natural nucleosides result in DNA chain termination events. NRTIs that are currently available include zidovudine (AZT), didanosine (ddI), zalcitabine (ddC), stavudine (d4T), lamivudine (3TC)), abacavir (ABC), emtricitabine (FTC) and tenofovir and tenofovir disoproxil fumarate (TDF), the latter often being referred to as Nucleotide Reverse Transcriptase Inhibitors (NtRTIs).

The function of the reverse transcriptase enzyme is to convert the RNA of HIV into DNA. In this process deoxynucleoside triphosphates (dNTP) are coupled to the growing DNA chain. Chain stoppers such as the NRTIs are first converted to the triphosphate (TP) by cellular kinases. For example AZT, which was one of the first HIV RT inhibitors identified, is converted to AZT-TP and HIV-1 RT is subsequently able to use AZT-TP as an efficient alternative substrate in the building of the viral DNA. However, AZT-TP lacks a 3′OH necessary for further DNA elongation, thereby causing termination of the growing DNA chain following incorporation. The inverse process, namely the removal of chain nucleotides or the removal of the chain-terminating residue such as AZT, is mediated by pyrophosphate or nucleoside triphosphates, also takes place but to a far lesser extend. This inverse process is enhanced in HIV mutants which have increased capability to remove the chain-terminating residue with much greater efficiency than wild type RT. This mechanism is seen as the cause of resistance of mutated HIV against AZT or any of the other NRTIs as described, for example in Götte et al., Journal of Virology, 2000, pp. 3579-3585.

Because of this inverse process, the addition of pyrophosphate or nucleoside triphosphates to an in vitro RT test model results in reduced inhibitory activity of the NRTI tested, in particular when mutated RT is used. Some NRTIs, however, show only a minimal reduction of RT activity or with some NRTIs RT activity even stays at the same level.

Resistance of the HIV virus against currently available HIV drugs continues to be a major cause of therapy failure. This has led to the introduction of combination therapy of two or more anti-HIV agents usually having a different activity profile. Significant progress was made by the introduction of HAART therapy (Highly Active Anti-Retroviral Therapy), which has resulted in a significant reduction of morbidity and mortality in HIV patient populations treated therewith. HAART involves various combinations of NRTIs, NNRTIs and PIs. Current guidelines for antiretroviral therapy recommend such triple combination therapy regimen for initial treatment. However, these multidrug therapies do not completely eliminate HIV and long-term treatment usually results in multidrug resistance. In particular, half of the patients receiving anti-HIV combination therapy do not respond fully to the treatment, mainly because of resistance of the virus to one or more drugs used. It also has been shown that resistant virus is carried over to newly infected individuals, resulting in severely limited therapy options for these drug-naive patients.

Consequently, there is a continued need for new types of active ingredients for use in drug combinations that are effective against HIV. Providing new types of anti-HIV effective active ingredients, differing in chemical structure and activity profile therefore is a highly desirable goal to achieve. Reverse transcriptase remains an interesting target and inhibitors of this enzyme are an indispensable part of HAART combinations. Finding agents that block the functioning of this enzyme via a new mechanism are expected to provide an alternative to the currently used NRTIs and NNRTIs. In particular the latter suffer from mutations that cause cross-resistance along this whole class. But also the NRTIs, although to a lesser extend, face resistance due to mutations. The fact that NRTIs show less cross-resistance is explained by a more complicated interaction with RT compared to the NNRTIs which apparently all interact with the same binding pocket so that a mutation causing a structural change in this pocket results in all NNRTIs becoming ineffective.

It has been found that RT inhibitors which show increased activity when pyrophosphate or nucleoside triphosphates are added in an in vitro test model, belong to a new class of anti-HIV agents. As mentioned above, only a decrease or a staying at the same level of RT activity has been found so far. Consequently compounds showing such increased activity are believed to interact differently with the RT enzyme and therefore belong to a new class of RT inhibitors. The present invention therefore is aimed at finding compounds that belong to this new class of HIV inhibitors.

The present invention is aimed at methods to identify new types of HIV RT inhibitors, which may find use in anti-HIV therapy and in particular may find use in anti-HIV drug combinations. The invention further provides methods for screening of compounds in order to identify those compounds that belong to a new class of HIV RT inhibitors.

### SUMMARY OF THE INVENTION

The present invention provides a method for identifying ribonucleotide or pyrophosphate sensitive RT inhibitors comprising:
a) providing a reaction well comprising
   at least one template for an HIV RT enzyme,
   at least one primer,
   at least one detectable dNTP substrate,
   at least one test compound;
   at least one RT enzyme, wherein said HIV RT enzyme incorporates the detectable dNTP substrate; and
determining RT activity by measuring the amount of the detectable dNTP substrate incorporated into the template;
b) providing another reaction well comprising
   at least one template for an HIV RT enzyme,
   at least one primer,
   at least one detectable dNTP substrate,
   at least one test compound;
   at least one nucleoside phosphate or at least one pyrophosphate,
   at least one RT enzyme, wherein said HIV RT enzyme incorporates the detectable dNTP substrate; and
determining RT activity by measuring the amount of the detectable dNTP substrate incorporated into the template;
c) comparing the RT activity obtained in step a) and in step b);
d) selecting the test compound wherein the RT inhibitory activity obtained in b) exceeds the RT inhibitory activity obtained in a);
   wherein the amount of the HIV RT inhibitor in steps a) and e) is the same and is such that an increase of RT activity is measurable.

Steps a) and b) in the method of the present invention may be conducted sequentially or in parallel. When conducted sequentially, step a) may be conducted first followed by step b) or vice versa.

In one embodiment the RT activity determined in steps a) and b) is a certain percent Inhibitory Concentration. In preferred embodiments the RT activity determined in steps a) and b) is an IC₅₀ or IC₉₀ value.

The invention also provides for a kit for identifying nucleotide competitive RT inhibitors, which comprises a template for an HIV RT enzyme; a primer, a detectable dNTP substrate; HIV RT enzyme; and a nucleoside phosphate, e.g. chosen from ATP and GTP, or a pyrophosphate.

In a further aspect the invention provides a method for identifying nucleotide competitive, ribonucleotide or pyrophosphate sensitive RT inhibitors comprising
a) providing a reaction well comprising
   at least one template for an HIV RT enzyme,
   at least one primer,
   at least one detectable dNTP substrate,
   at least one test compound;
   at least one RT enzyme, wherein said HIV RT enzyme incorporates the detectable dNTP substrate; and
determining RT activity by measuring the amount of the detectable dNTP substrate incorporated into the template;
b) providing another reaction well comprising
   at least one template for an HIV RT enzyme,
   at least one primer,
   at least one detectable dNTP substrate,
   at least one test compound;
   at least one nucleoside phosphate or at least one pyrophosphate,
   at least one RT enzyme, wherein said HIV RT enzyme incorporates the detectable dNTP substrate; and
determining RT activity by measuring the amount of the detectable dNTP substrate incorporated into the template;
c) comparing the RT activity obtained in step a) and in step b);
d) subjecting the test compound to a kinetic RT enzymatic assay;
e) selecting the test compound wherein the RT inhibitory activity obtained in b) exceeds the RT inhibitory activity obtained in a) and additionally that is competitive in the kinetic enzymatic assay of step d);
   wherein the amount of the HIV RT inhibitor in steps a) and b) is the same and is such that an increase of RT activity is measurable.

In the former method, steps a), b) and d) may be conducted in parallel or sequentially.

The present invention additionally is directed to various combinations and subcombinations of any of the features disclosed in this specification and claims.

### DETAILED DESCRIPTION OF THE INVENTION

In the methods of this invention the nucleoside phosphate includes ribonucleoside phosphates, ribonucleoside diphosphates and ribonucleoside monophosphates and may also include deoxyribonucleoside triphosphates, deoxyribonucleoside diphosphates and deoxyribonucleoside monophosphates as well as derivatives thereof such as the phosphates of ribonucleoside thio or imino derivatives. The ribonucleoside triphosphates may be chosen from ATP, GTP, UTP, CTP, the deoxyribonucleoside triphosphates may be chosen from dATP, dGTP, dUTP, TTP, dCTP. The ribonucleoside mono or diphosphates may be chosen from AMP, ADP, GMP, GDP, UMP, UDP, CMP, CDP. The deoxyribonucleoside mono or diphosphates may be chosen from dAMP, dADP, dGMP, dGDP, dUMP, dUDP, TDP, TMP, dCMP, dCDP. The phosphates of ribonucleoside thio or imino derivatives include for example ATPbgNH (adenosine 5′(beta,gamma,imido)triphosphate), ATPgS (adenosine 5′[gamma-thio]triphosphate). Of particular interest are ADP, AMP, ATPbgNH, ATPgS, ATP, CTP, GTP, UTP. Where the nucleoside phosphate is a deoxyribonucleoside triphosphate, the detectable dNTP substrate preferably is derived form another nucleic acid. Preferred for use in the invention are ATP or GTP, most preferred is ATP. The pyrophosphate or PPi may be a pyrophosphate salt such as an alkalimetal pyrophosphate, in particular sodium pyrophosphate.

In the methods of this invention, the ingredients of step a) and of step b) may be added to the test well in any given sequence. They may be added one by one or group wise such as combined in a mixture. In particular, the RT enzyme may be added first to the reaction well and then the other components are added or the other components may be added first, where after the RT enzyme is added to the reaction well. Also possible is that one or more of the components are added and then the RT enzyme, followed by the remaining components.

Thus in one embodiment, the assay provides a reaction well comprising a template for an HIV RT enzyme, a primer, a detectable dNTP substrate and a test compound. A HIV RT enzyme is then added to the reaction well, wherein the HIV RT enzyme incorporates the detectable dNTP substrate into the template. RT inhibitory activity of the test compound is measured. The test compound is subjected to another test in which another reaction well is provided comprising a template for an HIV RT enzyme, a primer, a detectable dNTP substrate, the test compound and a nucleoside phosphate, such as ATP and GTP, or a pyrophosphate. A wild type HIV RT enzyme is then added to the reaction well, wherein the HIV RT enzyme incorporates the detectable dNTP substrate into the template. RT inhibitory activity of the test compound in the presence of the nucleoside phosphate or the pyrophosphate is measured and compared with that obtained in the test without the nucleoside phosphate or the pyrophosphate. Those compounds are selected wherein the RT inhibitory activity of the test compound in the presence of the nucleoside phosphate or the pyrophosphate exceeds the RT inhibitory activity obtained in the test without the nucleoside phosphate or the pyrophosphate.

In another embodiment of the invention, the assay is conducted such that the HIV RT enzyme is present in a reaction well and the template, primer, detectable dNTP substrate, HIV RT inhibitor, and, in the second part of the assay the nucleoside phosphate or the pyrophosphate, are added to the HIV RT enzyme.

In the methods including a kinetic RT enzymatic assay, steps a), b) and d) may be conducted in parallel or sequentially, meaning that any combination of parallel or sequential runs can be done. For example all three steps can be run in parallel or all three steps can be run sequentially in any given sequence, e.g. step a), b) and d) sequentially, or d), b) and a) sequentially etc. Two steps may be run in parallel, while the other preceeds or follows these steps, e.g. a) and b) in parallel followed by d).

The methods of the present invention are based on a change in susceptibility of the RT enzymatic activity to a certain test compound. Susceptibilities can be generally expressed as ratios of IC₅₀ or IC₉₀ values of RT enzymatic activity in the presence and in the absence of a nucleoside phosphate, such as ATP or GTP, or a pyrophosphate. Ratios of other IC percentages are also possible. Susceptibilities are generally expressed as ratios of IC₅₀ or IC₉₀ values.

The IC₅₀ or IC₉₀ value is the test compound concentration at which 50% or 90% respectively of the enzymatic activity is inhibited. These values are determined using standard procedures.

The ratio of the IC value of RT enzymatic activity measured in the presence a nucleoside phosphate, e.g. ATP and GTP, or a pyrophosphate to the IC value of RT enzymatic activity measured in the presence a nucleoside phosphate, e.g. chosen from ATP and GTP, or a pyrophosphate should greater than 1, preferably said ratio should be greater than 3, more preferably it should be greater than 5. In particular said ratio is the ratio of the IC₅₀ or IC₉₀ values.

The kinetic RT enzymatic assay as in step d) is used to determine whether or not a test compound is a competitive RT inhibitor. It is an enzymatic kinetics assay in which the mechanism of inhibition of the HIV RT inhibitor is determined from the kinetics using a wild type HIV RT or a mutant RT protein. For example, the Michaelis constant, Km, the dissociation constant of the enzyme-inhibitor complex, Ki, and the mechanism of inhibition may be determined by fitting the data at various concentrations of substrate, RT inhibitor, and/or other reagents, to the Michaelis-Menten competitive inhibition equation, the Michaelis-Menten non-competitive inhibition equation and the Michaelis-Menten uncompetitive inhibition equation. If the best fit is obtained using the Michaelis-Menten competitive inhibition equation, than the inhibitor is a nucleotide-competitive RT inhibitor. Any other kinetic analysis known in the art may be utilized with the methods of the invention depending on the application envisaged.

The invention provides an in vitro, fast, and inexpensive method for detecting HIV RT inhibitors belonging to a new class.

In one embodiment, compounds, which are nucleoside triphosphates are discarded. These comprise any of the triphosphates of natural nucleosides or of derivatives thereof such as the NRTI triphosphates, in particular these comprise any triphosphates that compete with the natural nucleoside triphosphates for incorporation into elongating viral DNA by reverse transcriptase. In a particular embodiment, any nucleoside phosphate (nucleotide), either of natural nucleosides or of derivatives thereof, including mono-, di- or triphosphates, is excluded

The methods of the invention are especially applicable in high throughput testing or evaluation devices. It is within the practice of the invention, however, to prepare a sample rack or solid support made up of numerous reaction wells, such that each reaction remains isolated form one another. Simultaneous transfer of one or more reagents to the reaction wells may then be achieved by one of the many techniques used in the art of high throughput analysis.

One or more of the contents of each of the reaction wells may be varied. For example, in one embodiment, each reaction well contains a template for an HIV RT inhibitor, a primer, a detectable dNTP substrate and a HIV RT enzyme, which can be wild-type RT enzyme or a mutant RT enzyme. A nucleoside phosphate, e.g. chosen from ATP or GTP of a pyrophosphate is or is not added to each reaction well. The reaction wells may form an array or may employ another means of identifying or addressing each compartment. The RT activity of each reaction well may then be automatically determined from the amount of detectable dNTP substrate incorporated into the template of each reaction well, and recorded. Other embodiments include, but are not limited to varying the concentration of one or more of the components, the RT enzyme, and varying the nucleoside phosphate or pyrophosphate.

There are numerous methods for handling high throughput. i.e., analyzing a large number of samples in a relatively short period of time. Any method of high throughput analysis available may be applied to the methods of the invention. Examples include, but are not limited to: U.S. Pat. No. 5,985,215 of Sakazume et al., entitled ‘Analyzing Apparatus Having a Function Pipette Samples’; U.S. Pat. No. 6,046,056 of Parce et al., entitled ‘High Throughput Screening Assay Systems in Microscale Fluidic Devices’; WO 00/14540 of Pauwels et al., entitled ‘Method For the Rapid Screening of Analytes’; WO 99/30154 of Beutel et al., entitled ‘Continuous Format High Throughput Screening’; and WO 99/67639 of Wada et al., entitled ‘High Throughput Methods, Systems and Apparatus for Performing Cell Based Screening Assays’.

In the practice of the invention, any template that would serve as an effective template for an HIV RT enzyme may be used. The template may or may not be bound to the reaction well, but in a preferred embodiment is bound to the reaction well. In a further preferred embodiment the template is chosen from poly-rA or a heteropolymer RNA or DNA. Any primer complementary to the template chosen may be used. In one embodiment, the primers are chosen from oligo-dT or a primer complementary to the heteropolymer template.

Detectable dNTP substrates useful in the practice of the invention include any dNTP substrate, and in a preferred embodiment, any dTTP substrate (deoxythymidine triphosphate), that is detectable before and/or after integration into the template. Detectable dNTP substrates include but are not limited to a radioactive labeled dTTP or any radioactive labeled dNTP, and a dNTP substrate that is capable of being detected by fluorescence, luminescence, or absorption spectrometry. The detectable dNTP substrate may be detectable on its own or it may bind to a tracer, which may then be detected. The tracer may be an optical tracer, such as a tracer that may be detected by fluorescence, luminescence, or absorption spectrometry, or the tracer may be a radioactive labeled tracer. In one embodiment, the detectable dNTP substrate is bromo-deoxyuridine-triphosphate and the optical tracer is an antibody or a monoclonal antibody such as monoclonal anti-BrdU antibody, conjugated to alkaline phosphatase that binds to the dNTP substrate.

The test compounds for use in the methods of this invention can be any natural, natural derived or man-made materials. As used in this context the term ‘test compound’ refers to a single compound or to a mixture of compounds.

The reaction wells for use in the methods of the invention may or may not at least one nucleoside phosphate, e.g. chosen from ATP and GTP, or at least one pyrophosphate. The concentrations of ribonucleotides or pyrophosphates may be varied depending on the application envisaged. For example, the influence of pyrophosphate (together with ATP) in vivo obviously depends on the intracellular concentrations. In a preferred embodiment, the intracellular concentrations, which are well established at 3.2±1.5 mM for ATP, 0.5±0.2 mM for GTP, and 130 μM for pyrophosphate are used.

The methods of the invention may be conducted using wild-type RT enzyme or mutant RT enzyme. Any RT enzyme or mutant RT enzyme that may incorporate the detectable dNTP substrate into the template may be useful in the practice of the invention.

The invention also provides for a kit. The kit may be used for any of the methods described herein, in particular the kit may be used to select nucleotide-competitive RT inhibitors. The kit of the invention may comprise a template for an HIV RT enzyme; a primer; a detectable dNTP substrate; and a nucleoside phosphate, e.g. chosen from ATP and GTP, or a pyrophosphate. The kit may further comprise a mutant RT enzyme and/or a wild type RT enzyme.

The methods according to the present invention allow the detection of compounds that are RT inhibitors, which belong to a new class, namely RT inhibitors that show increased RT inhibitory activity in the presence of a nucleoside phosphate or a pyrophosphate. This new class of RT inhibitors differs from the existing RT inhibitors, i.e. the compounds of this class do not belong neither to the class of NRTIs nor to the NNRTIs. The latter are the class of compounds designated in the art as ‘non-nucleoside reverse transcriptase inhibitors’, which usually are meant to comprise those compounds, which interact with the so-called NNRTI ‘pocket’ in the RT enzyme. Without wishing to be bound by the following theory, it is assumed that compounds that are detected by the methods of this invention interact at the same location in the RT enzyme as the NRTIs, while the nucleoside phosphate (such as ATP) or the pyrophosphate also bind to the RT enzyme in such way that the functioning of the enzyme is blocked. This results in a double blocking of the enzyme so that compounds detected by the methods of this invention are believed to be very effective blockers of the HIV RT enzyme. Even without co-administration of a nucleoside phosphate or of a pyrophosphate this effect may play a role because nucleoside phosphates such as ATP are present in cell plasma.

Neither compounds belonging to the art-known classes of NRTIs nor NNRTIs show the increase of RT inhibiting activity as in the new class of RT inhibitors that can be identified using the methods of the present invention. Compounds that are detected by the methods of this invention therefore interact with the RT enzyme via a unique mechanism of action and will be active against HIV mutants that escape NRTI or NNRTI single or combined therapy. Such compounds therefore may find use in the treatment of patients that have been pre-treated with NRTIs and/or NNRTIs or in combination therapy.

In certain embodiments, the methods according to the present invention also allow the detection of compounds that are RT inhibitors that show increased RT inhibitory activity in the presence of a nucleoside phosphate or a pyrophosphate and moreover compete with the natural nucleoside triphosphates. Such methods allow the finding of compounds that have an NRTI like behavior but are chemically different, which is expected to result in drugs that are less sensitive to cross-resistance and that select different mutations. Such compounds could find use as alternatives for NNRTIs and/or NRTIs in drug cocktails. This type of compounds that are RT inhibitors, which belong to a new class, may be referred to as “nucleotide competitive RT inhibitors (NCRTIs)”.

All references, patents, and patent application cited herein are incorporated by reference in their entirety.

The following examples are provided by way of illustration and are not intended to be limiting of the present invention.

### EXAMPLES

### Example 1

In vitro inhibition of HIV reverse transcriptase in presence and absence of ATP.

The assay was run using kit TRK 1022™ (Amersham Life Sciences) according to the manufacturer's instructions with slight modifications. Test compounds were diluted in steps of ¼ in 100% DMSO and subsequently diluted 1/50 in Medium A (RPMI 1640+10% fetal calf serum, 20 mg/ml gentamycin).

In each experiment three conditions were tested: wells filled with 25 μl of the above compound solutions, wells filled with 25 μl 2% DMSO in Medium Λ (R0) and wells filled with 100 μl stop solution and 25 μl DMSO in Medium A (R1).

To each well was added 25.5 μL master mix (5 μL primer/template beads, 10 μl assay buffer, 0.5 μl tracer (50 μM [3H]-dTTP), 5 μL HIV RT enzyme solution (15 mU/μl), 5 μl Medium Λ). The plates were sealed, and incubated during 4 hours at 37° C. Subsequently, 100 μl stop solution was added to each well (except R1). The radioactivity was counted in a TopCount™.

For testing compound inhibition in the presence of ΛTP the same protocol as above was used but the Medium A in the master mix was replaced with Medium A containing 320 mM ΛTP.

Using the assay above, the IC₅₀ of compound 1 in the absence of ATP is 0.3 μM while the IC₅₀ of compound 1 in the presence of ATP is 0.016 μM.

Compound 1 has been described in WO-04/046143 is the compound having the structure

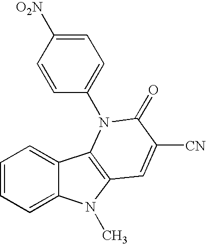

### Example 2

The procedures of example 1 were repeated but ATP was changed by a number of other nucleoside phosphates. The following table lists the tested nucleoside phosphates and the IC₅₀ values in μM of compound 1 obtained in the presence of the concerned nucleoside phosphate.

ATPgS is adenosine 5′[gamma-thio]triphosphate[CAS 93839-89-5]; and

ATPbgNH is adenosine 5′(beta,gamma,imido)triphosphate [CAS 72957-42-7].

| Nucleoside phosphate | IC₅₀ in μM |
|---|---|
| ADP | 0.023 |
| AMP | 0.0178 |
| ATPbgNH | 0.0245 |
| ATPgS | 0.0124 |
| CTP | 0.114 |
| GTP | 0.0704 |
| UTP | 0.0519 |
| PPi | 0.1992 |

### Example 3

### Enzyme Kinetics Studies

The enzyme kinetics studies were carried out using a protocol involving a 4×5 matrix of varying substrate and inhibitor concentrations over ranges of 40-3 μM of dTTP and 2-0 μM of compound 1.

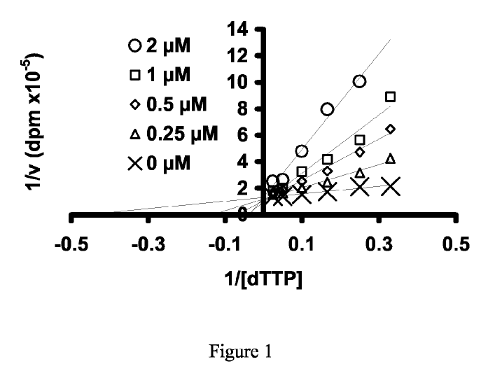

The reaction mixtures (50 μl) further contained 50 mM Tris.HCl (pH 7.8), 5 mM dithiothreitol, 300 mM glutathione, 500 μM EDTA, 150 mM KCl, 5 mM MgCl₂, 0.15 mM of the template/primer poly(rA)oligo(dT) and 0.06% Triton X-100. The reaction mixtures were incubated at 37° C. for 15 min, at which time 100 μl of calf thymus DNA (150 pg/ml), 2 ml of Na₄P₂0₇ (0.1 M in 1 M HCl), and 2 ml of trichloroacetic acid (10% v/v) were added. The solutions were kept on ice for 30 min, after which the acid-insoluble material was washed and analyzed for radioactivity. When the reciprocal of the reaction velocity (1/v) is plotted against the reciprocal of the substrate concentration (1/[dTTP]) in the presence of different concentrations of compound 1, the graph (See FIG. 1) is obtained.

Since all the lines cross at the intercept with the Y-axis it is clear that this compound has a competitive mode of HIV RT inhibition.

1. A method for identifying ribonucleotide or pyrophosphate sensitive RT inhibitors comprising:
a) providing a reaction well comprising
at least one template for an HIV RT enzyme,
at least one primer,
at least one detectable dNTP substrate,
at least one test compound;
at least one RT enzyme, wherein said HIV RT enzyme incorporates the detectable dNTP substrate; and
determining RT activity by measuring the amount of the detectable dNTP substrate incorporated into the template;
b) providing another reaction well comprising
at least one template for an HIV RT enzyme,
at least one primer,
at least one detectable dNTP substrate,
at least one test compound;
at least one nucleoside phosphate or at least one pyrophosphate,
at least one RT enzyme, wherein said HIV RT enzyme incorporates the detectable dNTP substrate; and
determining RT activity by measuring the amount of the detectable dNTP substrate incorporated into the template;
c) comparing the RT activity obtained in step a) and in step b)
d) selecting the test compound wherein the RT inhibitory activity obtained in b) exceeds the RT inhibitory activity obtained in a);
wherein the amount of the HIV RT inhibitor in steps a) and e) is the same and is such that an increase of RT activity is measurable.

2. A method for identifying nucleotide competitive, ribonucleotide or pyrophosphate sensitive RT inhibitors comprising
a) providing a reaction well comprising
at least one template for an HIV RT enzyme,
at least one primer,
at least one detectable dNTP substrate,
at least one test compound;
at least one RT enzyme, wherein said HIV RT enzyme incorporates the detectable dNTP substrate; and
determining RT activity by measuring the amount of the detectable dNTP substrate incorporated into the template;
b) providing another reaction well comprising
at least one template for an HIV RT enzyme,
at least one primer,
at least one detectable dNTP substrate,
at least one test compound;
at least one nucleoside phosphate, e.g. chosen from ATP and GTP or at least one pyrophosphate,
at least one RT enzyme, wherein said HIV RT enzyme incorporates the detectable dNTP substrate; and
determining RT activity by measuring the amount of the detectable dNTP substrate incorporated into the template;
c) comparing the RT activity obtained in step a) and in step b);
d) subjecting the test compound to a kinetic RT enzymatic assay;
e) selecting the test compound wherein the RT inhibitory activity obtained in b) exceeds the RT inhibitory activity obtained in a) and additionally that is competitive in the kinetic enzymatic assay of step d);
wherein the amount of the HIV RT inhibitor in steps a) and e) is the same and is such that an increase of RT activity is measurable.

3. The method of claim 1 wherein steps a) and b) are conducted sequentially.

4. The method of claim 1 wherein steps a) and b) are conducted in parallel.

5. The method of claim 2 wherein steps a) and b) are conducted sequentially followed by step d).

6. The method of claim 2 wherein steps a) and b) are conducted in parallel followed by step d).

7. The method of claim 1 wherein the RT activity determined in steps a) and b) is a certain percent Inhibitory Concentration, in particular an IC₅₀ or IC₉₀ value.

8. The method of claim 1 wherein the nucleoside phosphate is selected from ATP and GTP.

9. A kit for identifying nucleotide competitive RT inhibitors, which comprises a template for an HIV RT enzyme; a primer, a detectable dNTP substrate; and a nucleoside phosphate or a pyrophosphate.
